(19) United States
(12) Patent Application Publication
(10) Pub. No.: US 2026/0060643 A1
(43) Pub. Date: Mar. 5, 2026

(54) RHEUMATIC HEART DISEASE DETECTION FROM ECHOCARDIOGRAMS

(71) Applicant: CHILDREN'S NATIONAL MEDICAL CENTER, Washington, DC (US)

(72) Inventors: Marius George LINGURARU, Washington, DC (US); Pooneh ROSHANITABRIZI, Washington, DC (US); Craig SABLE, Washington, DC (US)

(73) Assignee: CHILDREN'S NATIONAL MEDICAL CENTER, Washington, DC (US)

(21) Appl. No.: 19/108,200

(22) PCT Filed: Sep. 15, 2023
(86) PCT No.: PCT/US2023/032933

§ 371 Date: Mar. 3, 2025

Related U.S. Application Data
(60) Provisional application No. 63/375,891, filed on Sep. 16, 2022.

(51) Int. Cl. A61B 8/08; A61B 8/00

(52) U.S. Cl. CPC A61B 8/0883; A61B 8/488; A61B 8/5223; A61B 8/5246; A61B 8/5292

(57) ABSTRACT

A method for detecting rheumatic heart disease (RHD) based on at least an echocardiogram, the method including receiving echocardiogram data, extracting first frames corresponding to at least one echocardiogram view from the echocardiogram data, extracting second frames corresponding to ventricular systole from the first frames corresponding to the at least one echocardiogram view, and determining, via at least one machine learning model, an RHD risk score based on the second frames corresponding to ventricular systole.

### CROSS-REFERENCE TO RELATED APPLICATIONS

The present application claims priority to U.S. Provisional Application No. 63/375,891, filed Sep. 16, 2022, which is incorporated herein by reference in its entirety for all purposes.

### BACKGROUND OF THE INVENTION

### Field of the Disclosure

The present disclosure is related to automated detection of rheumatic heart disease (RHD).

### Description of the Related Art

RHD often presents as mitral valve regurgitation (MR), or blood backflow in the left atrium of the heart during ventricular systole (contraction). Detection of RHD involves both a spatial and temporal analysis of echocardiograms in order to properly identify and assess MR.

The foregoing Background description is for the purpose of generally presenting the context of the disclosure. Work of the inventors, to the extent it is described in this background section, as well as aspects of the description which may not otherwise qualify as prior art at the time of the filing, are neither expressly or impliedly admitted as prior art against the present disclosure.

### SUMMARY OF THE INVENTION

The foregoing paragraphs have been provided by way of general introduction, and are not intended to limit the scope of the following claims. The described embodiments, together with further advantages, will be best understood by reference to the following detailed description taken in conjunction with the accompanying drawings.

In one embodiment, the present disclosure is related to a method for detecting rheumatic heart disease (RHD) based on at least an echocardiogram, the method comprising: receiving, via processing circuitry, echocardiogram data; extracting, via the processing circuitry, first frames corresponding to at least one echocardiogram view from the echocardiogram data; extracting, via the processing circuitry, second frames corresponding to ventricular systole from the first frames corresponding to the at least one echocardiogram view; and determining, via at least one machine learning model executed by the processing circuitry, an RHD risk score based on the second frames corresponding to ventricular systole.

In one embodiment, the present disclosure is related to a non-transitory computer-readable storage medium for storing computer-readable instructions that, when executed by a computer, cause the computer to perform a method for detecting rheumatic heart disease (RHD) based on at least an echocardiogram, the method comprising: receiving echocardiogram data; extracting first frames corresponding to at least one echocardiogram view from the echocardiogram data; extracting second frames corresponding to ventricular systole from the first frames corresponding to the at least one echocardiogram view; and determining, via at least one machine learning model, an RHD risk score based on the second frames corresponding to ventricular systole.

In one embodiment, the present disclosure is related to an apparatus for detecting rheumatic heart disease (RHD) based on at least an echocardiogram, comprising: processing circuitry configured to receive echocardiogram data, extract first frames corresponding to at least one echocardiogram view from the echocardiogram data, extract second frames corresponding to ventricular systole from the first frames corresponding to the at least one echocardiogram view, and determine, via at least one machine learning model, an RHD risk score based on the second frames corresponding to ventricular systole.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a method for detecting RHD from echocardiograms, according to one embodiment of the present disclosure;

FIG. 2 is a schematic of a view identification model, according to one embodiment of the present disclosure;

FIG. 3 is a schematic of an RHD detection model, according to one embodiment of the present disclosure;

FIG. 4 is a schematic of an RHD classification network, according to one embodiment of the present disclosure;

FIG. 5 is a method for detecting RHD from echocardiograms, according to one embodiment of the present disclosure;

FIG. 6 is a schematic of multi-view 3D convolutional neural networks, according to one embodiment of the present disclosure;

FIG. 7 is a method for detecting and characterizing MR jets and RHD from echocardiograms, according to one embodiment of the present disclosure; and

FIG. 8 is a schematic of a hardware configuration of a device for performing a method, according to one embodiment of the present disclosure.

### DETAILED DESCRIPTION OF THE PREFERRED EMBODIMENTS

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

RHD is a common acquired heart condition in young children and is treatable if detected early. RHD can be detected via echocardiograms, which are acquired via ultrasound and can show blood flow in the heart. Abnormal blood flow, such as mitral valve regurgitation (MR), can be indicative of RHD. MR occurs when a damaged mitral valve closes during the ventricular systole phase of the cardiac cycle. The damage to the mitral valve from RHD can result in the valve not being able to close tightly during contraction, resulting in blood flowing backwards from the left ventricle into the left atrium. The visual representation of leakage or backflow of blood into the left atrium due to MR in an echocardiogram can be termed an MR jet. The magnitude and volume of blood backflow can affect the visual characteristics (e.g., color and size) of the MR jet. Analysis of echocardiograms to detect RHD is typically performed by expert cardiologists. For instance, features of an MR jet, such as the dilation, duration, and blood velocity, can be assessed based on an echocardiogram in order to determine the severity of MR and corresponding RHD. However, there are many environments where these experts are not readily available to assess each echocardiogram that is collected from a patient population.

An echocardiogram is a dynamic representation of blood flow. Thus, the appearance of an MR jet in an echocardiogram changes over time as the heart beats. Cardiac gating can synchronize timing of image acquisition in order to capture an echocardiogram at a known point in the cardiac cycle (e.g., ventricular systole). However, cardiac gating is not always available. For instance, low-cost, handheld ultrasounds, which are used in areas where RHD is prevalent, are often not equipped with gating functionality. The echocardiograms collected by these ultrasounds can include a large amount of data over multiple views of the heart and multiple heart beats. A lack of time-synchronized data results in a need for additional analysis of echocardiograms in order to locate the mitral valve and identify when ventricular systole is occurring before an MR jet and RHD can be properly detected.

Therefore, there is a need for systems and methods for automated analysis of echocardiograms in order to detect RHD. An automated prediction method, as presented herein, can include a combination of deep learning and machine learning frameworks to harmonize a series of frames of an echocardiogram, identify and characterize MR jet region(s), and predict whether a patient has RHD and/or a classification of RHD (e.g., definite, borderline) based on the echocardiogram. The systems and methods described in this disclosure can be especially useful in low-resource settings where patients do not have access to specialized medical facilities, equipment, or personnel. In one embodiment, the automated methods can provide a diagnosis of RHD as well as additional visualization and characterization of cardiac activity (e.g., of an MR jet) for clinical analysis. In one embodiment, the cardiac activity can include aortic valve regurgitation (AR), which is a backflow of blood into the left ventricle through the aortic valve. AR can be visualized in a similar manner to MR. In one embodiment, the methods disclosed herein for characterization of an MR jet can be applied to characterization of AR or other cardiac activity that can be visualized on an echocardiogram. The automated detection of RHD, as described herein, can be performed based on at least an echocardiogram independently of or in addition to the characterization of an MR jet. In one embodiment, the methods of RHD detection can include receiving input of data, e.g., patient demographic and/or clinical data, and incorporating the patient data into the determination of RHD. In one embodiment, the methods of RHD detection can include receiving an input (e.g., a selection) related to any of the classification or analysis steps described herein in order to enhance performance.

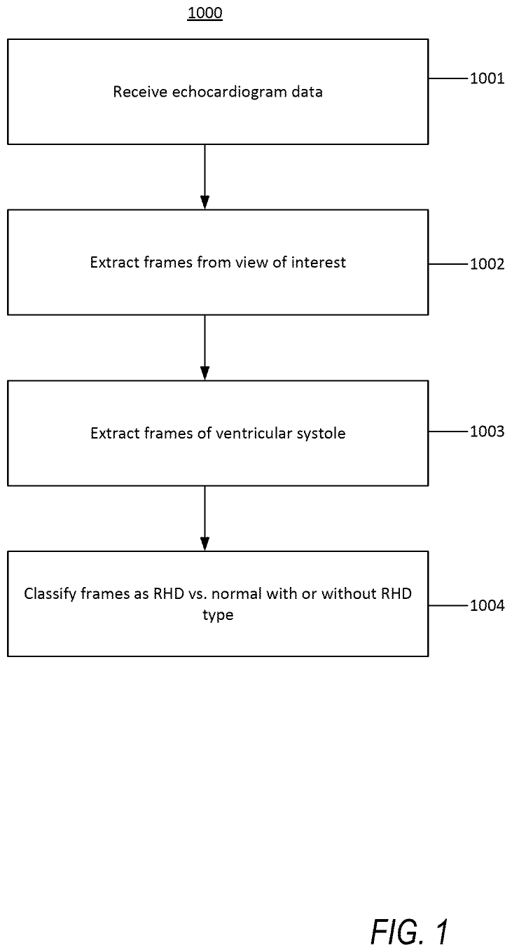

FIG. 1 is a flow chart of a method 1000 for detecting RHD based on echocardiograms, according to one embodiment of the present disclosure. Each step of the method 1000 will be described in further detail herein. The method 1000 can be executed by a computer or similar electronic device, including a server, a mobile phone, etc. The method can include receiving echocardiogram data collected by an ultrasound in step 1001. The echocardiogram data can include video data. In one embodiment, the method 1000 can be used to analyze Doppler echocardiograms with or without B-mode ultrasound, which can indicate the flow of blood through the heart. Specifically, in color Doppler echocardiograms, the direction of blood flow can be represented by the color of the echocardiogram. In step 1002, the echocardiograms can be analyzed to extract frames that are acquired from one or more views of interest. A view can refer to a certain position, angle, or orientation of the ultrasound probe during the acquisition of the echocardiogram. In step 1003, the frames from the one or more views of interest can be analyzed to extract frames corresponding to the ventricular systole phase of the cardiac cycle. In step 1004, the extracted frames from each view can be processed separately and/or together to classify the echocardiogram as being indicative of RHD or not. A label or probability of “RHD” or “normal” can be assigned to the echocardiogram. In one embodiment, a label and/or probability of RHD type (e.g., a severity, borderline, definite) can be assigned to the echocardiogram. In one embodiment, the extracted frames can be processed to assign an RHD risk score to the echocardiogram.

The method 1000 can be executed using one or more machine learning models (such as deep learning models with any structure or architecture). In one embodiment, two or more steps of the method can be combined in a single machine learning model. For example, the frame selection of step 1003 and the analysis of the extracted frames of step 1004 can be merged into a single model. The machine learning model(s) can be trained to perform the steps in sequence. In one embodiment, the one or more machine learning models can include a deep learning model with multiple layers, such as a neural network (e.g., convolutional neural network (CNN), residual neural network). A machine learning model, as referenced herein, can be trained to process, analyze, encode, and/or classify an input using training data. In one embodiment, a machine learning model can assign a binary label and/or a probability to an input. Training data can refer to data with a known characteristic or encoding. For example, training data in the present disclosure can refer to echocardiograms that are known to be either normal echocardiograms or RHD echocardiograms and can be labeled with a probability and/or a type of RHD.

The machine learning models can include one or more classifiers. The one or more classifiers can be used to determine whether echocardiogram data belongs to one or more categories (e.g., RHD, normal). In one embodiment, the one or more machine learning models can include an attention-based network. In an attention-based network, portions of the input can be dynamically assigned different weights indicating the relevance or importance of the portions for detecting RHD. For example, frames of the input that correspond to ventricular systole can be more relevant in detecting RHD because of, for example, the appearance of MR in those frames and can be assigned a greater weight. In one example, frames or regions of frames corresponding to the left atrium can be assigned a greater weight because of, for example, the appearance of MR in those regions.

A thorough evaluation of the heart using echocardiography requires capturing images from various angles and orientations (“views”) and in different modes. The images can be acquired over multiple heart beats and presented as a series of frames of echocardiogram data or a video. Each view of an echocardiogram can be captured over one or more frames and can provide data on different structures of the heart. It can therefore be helpful to isolate one or more views that are most relevant for assessing MR, as recited in step 1002 of FIG. 1. In one non-limiting embodiment, the step 1002 can include extracting frames, the frames for example corresponding to the apical 4-chamber (A4CC) view, the apical 5-chamber (A5CC) view, and/or the parasternal long-axis (PLAXC) view from a set of color Doppler echocardiogram data. The extraction of echocardiogram data from the A4CC and PLAXC views can account for differing appearance of the heart across different ultrasound transducer positions. The integration of the A4CC and PLAXC views can improve prediction accuracy compared to when each view is used independently.

The A4CC view frames and the PLAXC view frames can be identified and extracted using at least one view identification model. The view identification model can be any deep learning model comprising a plurality of layers, such as a Dense-Net-121 convolutional neural network (CNN). The input to the view identification model can be at least one representative frame (e.g., the first frame, all frames) of each view. The view identification model can be trained to classify whether or not the frame corresponds to a view of interest. In one example, the view identification model can be used to classify a frame as an A4CC view, a PLAXC view, or an “other” view (neither A4CC nor PLAXC).

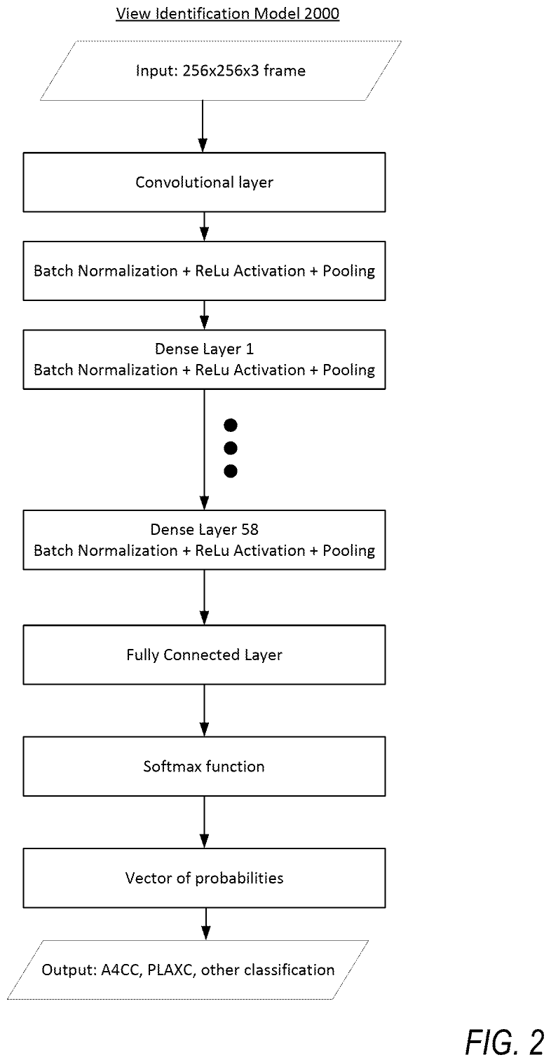

FIG. 2 shows an example schematic of a view identification machine learning model 2000, according to one embodiment of the present disclosure. According to one example, the view identification model can be a dense model, such as a Dense-Net-121 convolutional neural network (CNN). A dense model can refer to a model with one or more dense layers. A dense layer can be a fully connected layer that can receive each output from the previous layer. In one embodiment, the view identification model can include, for example, four dense blocks. A dense block can refer to a grouping of layers wherein each layer in the grouping of layers is connected to each other. As an illustrative example, in a dense block of five layers, the output from a first layer is input to the following second layer as well as to the third layer, the fourth layer, the fifth layer.

In one embodiment, the input frame of the view identification model 2000 can be, for example, a 256×256 pixel image with 3 color channels. The method 1000 can include resizing the echocardiograph data received in step 1001 to the input frame size of the view identification model 2000. In one embodiment, the first convolutional layer can include, for example, 64 filters. The view identification model 2000 can include, for example, 58 dense layers, wherein each dense layer can include, for example, a batch normalization (BN) step, an activation function (e.g., a rectifier (ReLu)), and a pooling layer. In one embodiment, the growth rate, or number of channels output by each layer, can be, for example, 32. The last fully connected layer can include, for example, three output neurons (A4CC neuron, PLAXC neuron, other neuron). The output of the last fully connected layer can be determined based on an activation or probability function, such as a Softmax probability function. A Softmax probability function can be used to convert a vector of k real numbers output by a neural network layer into a probability distribution of k possible outcomes, wherein k is an integer by applying an exponential function to each element in the vector. In one embodiment, the Softmax probability function can output a vector of probabilities of the input frame corresponding to a view. As an example, the view can be an A4CC view, PLAXC view, or other (neither the A4CC nor PLAXC views). The vector of probabilities can be based on the output of each of the three output neurons in the last fully connected layer.

In one embodiment, the view identification model 2000 can be trained to identify echocardiogram views using, for example, the Adam optimization algorithm with an example learning rate of 0.0001 and an example batch size of 4 for 100 epochs. In general, training of a machine learning model can involve running the machine learning model on training data and adjusting weights and other parameters of the machine learning model using an optimization algorithm in order to minimize a loss function. The loss function can quantify a difference between a known result and a predicted result. For example, the view identification model can be trained on echocardiograms from different views. The known result can be the actual view corresponding to an echocardiogram. The predicted result can be the view predicted by the view identification model 2000 based on, for example, the at least one representative frame of the echocardiogram or based on all frames of the echocardiogram. The view identification model 2000 can be any deep learning model and is not limited to the architecture or model type illustrated in FIG. 2. Minimizing the loss function can minimize the difference between the known result and the predicted result, thus resulting in increased accuracy of the model. In one embodiment, the minimized loss function can be, for example, the categorical cross-entropy loss function.

In one embodiment, the device executing the method 1000 can extract frames from, for example, the A4CC view and the PLAXC view based on the classification output of the view identification model. The A4CC and PLAXC echocardiograms can be used as example input(s) into at least an RHD detection model. The echocardiograms can be ungated or gated and can include ultrasound data collected over one or more heartbeats. In one embodiment, the A4CC view and the PLAXC view can either be treated as separate inputs or concatenated into a single input for at least an RHD detection model.

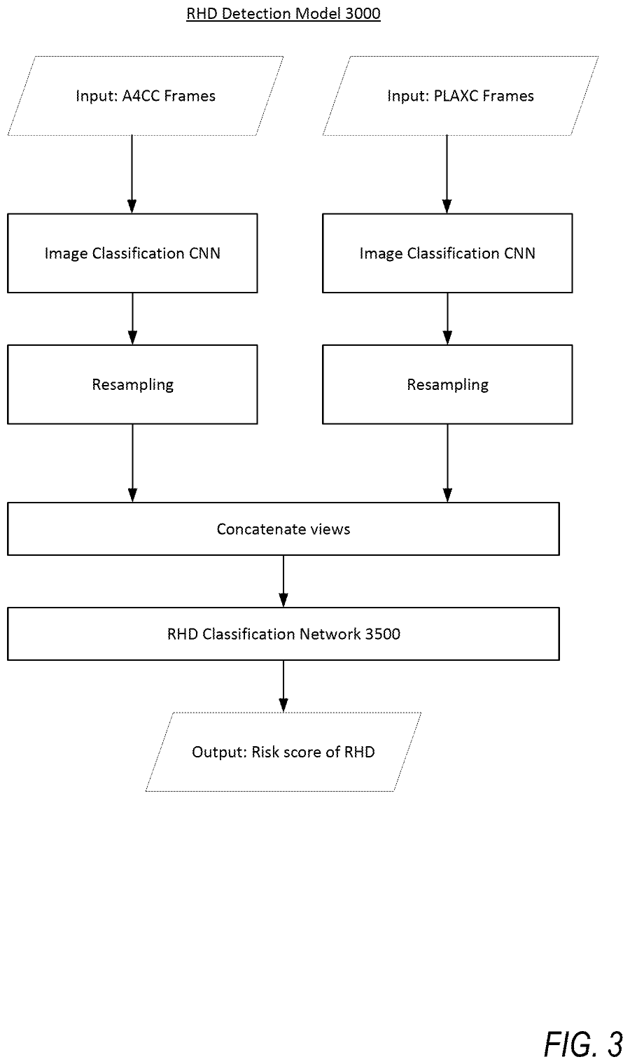

FIG. 3 is an example schematic of an RHD detection model 3000, according to one embodiment of the present disclosure. The RHD detection model can be used to analyze the frames from, for example, the A4CC view and the PLAXC view and can classify frames corresponding to a phase of the cardiac cycle, such as ventricular systole, from the input(s). Any phase of the cardiac cycle is compatible with the present disclosure and the description of the RHD detection model herein. For instance, the RHD detection model can be used to classify frames corresponding to diastole in order to weight frames that may show aortic valve regurgitation. The RHD detection model can then be used to process the frames corresponding to the phase of the cardiac cycle in order to predict whether the echocardiograms are indicative of RHD. In one embodiment, the RHD detection model of FIG. 3 can be used to execute steps 1003 and 1004 of the method 1000 in a single, end-to-end network. The RHD detection model can also be trained as a single, end-to-end network or trained as two separate networks. In one embodiment, the RHD detection model can be used to accurately predict RHD based on entire echocardiogram frames rather than based on localized regions within an echocardiogram frame.

In one embodiment, the RHD detection model can include, for example, a first set of image classification CNNs for analyzing each of the echocardiogram views to select frames from a phase in the cardiac cycle, as shown in FIG. 3. In one example, the frames can correspond to ventricular systole. Alternative and additional phases or segments of the cardiac cycle are compatible with the models and methods described herein. In one example, the image classification CNNs can be a residual neural network (ResNet) such as ResNet50. A residual neural network can include skip connections, wherein an input to a first layer can be merged with the output of a non-adjacent downstream layer for continued processing. The inclusion of skip connections can improve accuracy of networks over a larger number of layers. In one embodiment, the image classification CNNs can extract features from each frame over a plurality of spatial resolutions (e.g., five spatial resolutions). Image analysis over a plurality of spatial resolutions can result in more accurate feature detection and extraction. In one embodiment, the output layer of the first image classification CNN shown in FIG. 3 can include, for example, a Softmax probability function to determine whether an echocardiogram frame was acquired during ventricular systole. The device executing the method 1000 can then extract frames that correspond to ventricular systole for each view. In this manner, the RHD detection model can be an attention-based network, wherein frames corresponding to ventricular systole are given more weight (attention) via extraction in detecting RHD.

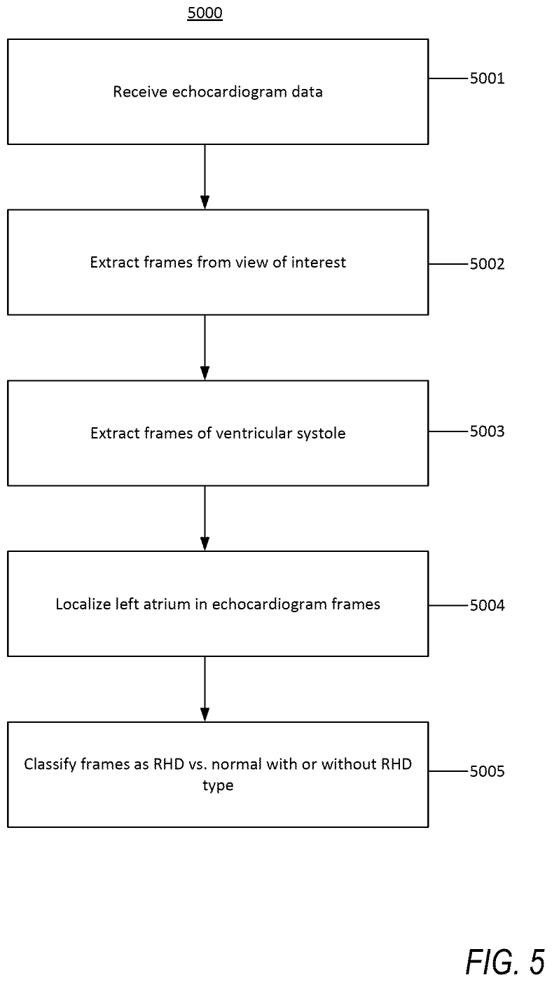

In one embodiment, a second image classification CNN or second set of image classification CNNs can localize atrium regions of the heart during ventricular systole, as described in greater detail with reference to FIG. 5. The first set of image classification CNNs and the second image classification CNN can have different, similar, or substantially the same architecture. In this manner, the RHD detection model can be an attention-based network, wherein frames corresponding to ventricular systole and/or localized atrium regions are given more weight (attention) via extraction in detecting RHD.

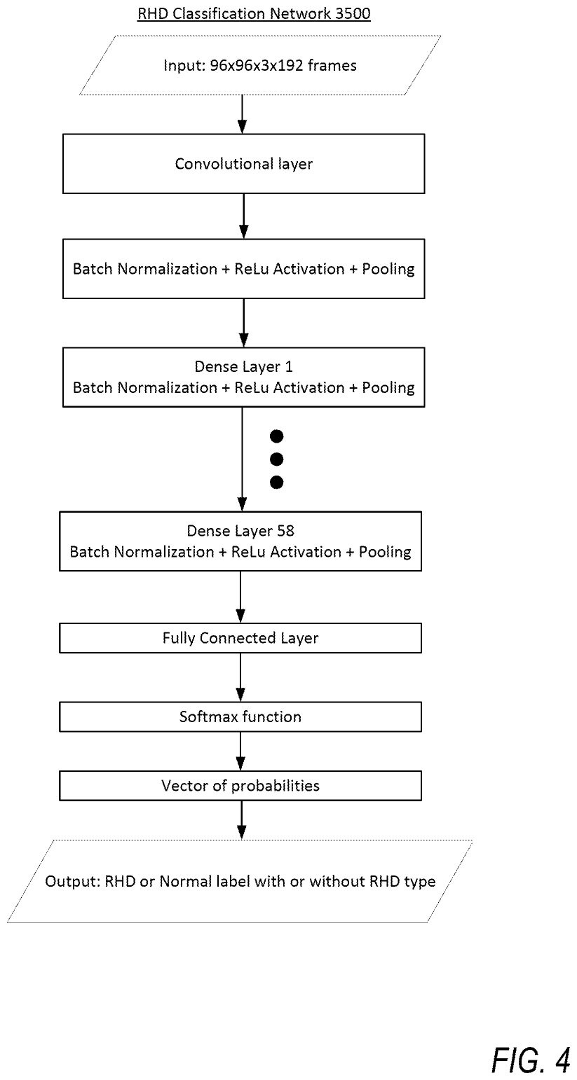

In one embodiment and with reference to FIG. 3, the extracted ventricular systole frames for each view can be concatenated to form a single input. According to one example, the concatenated input can include, for example, 96 frames from each view (e.g., A4CC, PLAXC) for a total of, for example, 192 frames, and each frame can be, for example, a 96×96 pixel image with 3 color channels. In one embodiment, the RHD detection model can include at least one RHD classification network 3500 for detecting RHD following the first image classification CNNs. The RHD classification network can be any deep learning model. In one example, the RHD classification network 3500 can be a dense network, e.g., a Dense-Net-121 CNN. FIG. 4 shows an example schematic of the RHD classification network 3500, according to one embodiment of the present disclosure. In one embodiment, the architecture of the RHD classification network 3500 can be different, similar to, or the same as the architecture of the view identification model 2000.

The RHD classification network can process the 4D concatenated input (for example, two-dimensional ventricular systole echocardiogram frames from A4CC and PLAXC views with color channels over time) to evaluate the input frames. The RHD classification network can then predict whether the patient has RHD based on the input frames. In one embodiment, the RHD classification network can include an activation function or probability function (e.g., Softmax function). In one embodiment, the output of the Softmax function can be used to assign a classification (label) and/or a probability of whether the input echocardiogram corresponds to a patient with RHD with or without an RHD type.

According to one example, the RHD detection model can be trained using, for example, the Adam optimization algorithm with an example batch size of 4 and an example scheduled learning rate of, for example, 130 epochs. In one embodiment, the total loss function Lₜₒₜₐₗ, for example, that is minimized during training can be calculated, for example, using the following equation (1):

$${L}_{total} = α {L}_{A 4 CC} ( {\hat{y}}_{A} , {y}_{A} ) + β {L}_{PLAXC} ( {\hat{y}}_{P} , {y}_{P} ) + γ {L}_{RHD} ( {\hat{y}}_{R} , {y}_{R} ) , \qquad ( 1 )$$

wherein L_{A4CC}, L_{PLAXC}, and L_{RHD} are, for example, the binary cross-entropy loss functions for, for example, A4CC and PLAXC frame selection and RHD detection, respectively, and y and y indicate the predicted result and original label, respectively. According to one example, the weights a, P, and y can be set to 1, 1, and 2. In one embodiment, the RHD detection model can be trained with an example initial learning rate of 0.001. The initial learning rate can be modified based on the loss over each epoch. For example, if the loss does not decrease by a certain amount (e.g., a threshold of 0.0001) for example three consecutive epochs, the initial learning rate can be reduced by a factor of example 0.1.

As an example, the RHD detection model can trained to label echocardiogram data as normal or as indicating RHD using labeled training data or provide the probability of RHD vs. normal with/without RHD type. The RHD detection model can then be evaluated for example for accuracy in labeling echocardiogram data that was not used for training. The hyperparameters yielding, for example, maximum accuracy for RHD detection during validation can be selected. The RHD detection model can be retrained once selected hyperparameters are set.

FIG. 5 is a flow chart of a method 5000 for detecting RHD based on echocardiograms by localizing an atrial region, such as the left atrium, according to one embodiment of the present disclosure. The method can include receiving echocardiogram data in step 5001. The echocardiogram data can include video data. In one embodiment, the method 5000 can be used to analyze Doppler or color Doppler echocardiograms with or without B-mode ultrasound, which can indicate the flow of blood through the heart. In color Doppler echocardiograms, the direction of blood flow can be represented by different colors. In step 5002, the echocardiograms can be analyzed to extract one or more views of interest, as is described herein with reference to step 1002 of method 1000. The view of interest can be, for example, the A4CC view and/or the PLAXC view. In step 5003, the echocardiograms from the one or more views of interest can be analyzed separately or together to extract frames corresponding to ventricular systole, as is described herein with reference to step 5003 of method 1000. In step 5004, the left atrium can be localized and/or segmented in the echocardiogram. In one embodiment, the extraction of echocardiogram frames and features in step 5003 or steps 5002 through 5004 can be referred to as echocardiogram homogenization. In step 5005, RHD can be detected based on the homogenized echocardiogram data. The homogenized echocardiogram data can refer to echocardiogram data from at least an A4CC view and/or a PLAXC view during ventricular systole. A label and/or probability of RHD with/without RHD type can be assigned to an echocardiogram as an output. The label can indicate whether the echocardiogram is indicative of the patient having RHD.

In one embodiment, the left atrium can be localized in echocardiogram frames corresponding to ventricular systole in step 5004 using a localization model and/or a segmentation model. The localization and/or segmentation model can be any machine learning model. The localization and/or segmentation model can include, for example, one or more convolutional layers followed by one or more dense (fully connected) layers. For example, the localization and/or segmentation model can include a LinkNet neural network with a VGG16 encoder backbone architecture. In one embodiment, each layer of the localization model except for the last (output) layer can include, for example, a ReLu activation function. The output layer can include, for example, a sigmoid probability function. In one embodiment, an input frame to the localization and/or segmentation model can be, for example, a 256×256 pixel echocardiogram frame with 3 color channels. The input frame can be for example at least one frame from an A4CC view and/or PLAXC view during ventricular systole that was extracted from the echocardiogram data during steps 5002 and 5003. The localization and/or segmentation model can be used to output a classification of regions (e.g., pixels) of the input frame that contain the left atrium. In one embodiment, the localization and/or segmentation model can be used to analyze the input image over a plurality of spatial resolutions (e.g., five spatial resolutions). In one example, the localization and/or segmentation model can be trained using, for example, the Adam optimization algorithm over example 500 epochs with an example batch size of 32 and an example learning rate of 0.0001. In one embodiment, the localization and/or segmentation model can be trained to, for example, minimize the negative value of the example Dice similarity coefficient as the loss function. The localization and/or segmentation model can be trained and used to localize additional or alternative regions of the heart, such as a ventricle (e.g., left ventricle), more than one chamber of the heart, a region within a chamber of the heart, etc.

RHD can be classified based on the homogenized echocardiogram data in step 5005 using, for example, an RHD ensemble model. The input to the RHD ensemble model can include homogenized echocardiograms of the localized left atrium regions captured during ventricular systole from, for example, the A4CC or/and PLAXC views. In one embodiment, the input to the RHD ensemble model can include homogenized echocardiograms of the frames during ventricular systole from, for example, the A4CC or/and PLAXC views without atrium localization. In one embodiment, the input to the RHD ensemble model can include homogenized echocardiograms of the frames from, for example, the A4CC or/and PLAXC views. In one embodiment, the homogenized echocardiograms for each view (e.g., A4CC, PLAXC) can be processed as separate inputs for at least a portion of the RHD ensemble model or concatenated into a single input. The RHD ensemble model can include different machine learning models that can be used to independently evaluate the homogenized echocardiogram data using different approaches. In one example, the RHD ensemble model can include at least one machine learning classifier with any structure such as multi-view, three-dimensional (3D) CNNs. The multi-view 3D CNNs can each be used to output a predictive RHD risk score using the same homogenized echocardiogram input. In one embodiment, the predictive RHD score(s) of classifier(s) can be fused in the RHD ensemble model using, for example, max-voting. The fusion can be done at different network depths, and/or prior to classification. In the example of max-voting, the predictive RHD score that is output the most (e.g., the mode) by the models is output as the final predictive RHD score.

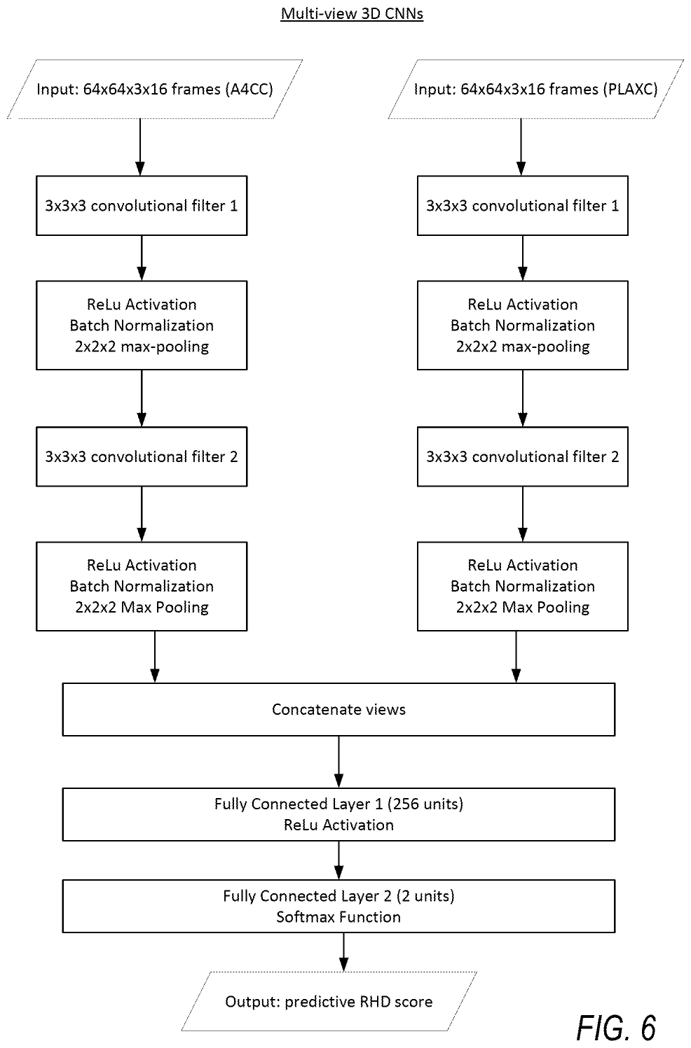

FIG. 6 is an example schematic of the multi-view 3D CNNs of the RHD ensemble model, according to one embodiment of the present disclosure. According to one embodiment, the input to the multi-view 3D CNNs can be a series of, for example, 16 frames for each view, and each frame can be, for example, a 64×64 pixel image with 3 color channels. In one embodiment, the multi-view 3D CNNs can include, for example, a first 3×3×3 convolutional filter and a second 3×3×3 convolutional filter. The stride size of the convolutional filters can be, for example, 1×1×1. Each convolutional filter can include, for example, a ReLu activation function. Each convolutional filter can be followed, for example, by a batch normalization layer and a 2×2×2 max pooling layer with stride length of 2 in each dimension. The example 16 frames for each view can be separately processed by the first and the second convolutional filter to extract features of each view.

In one embodiment, the outputs of the second max pooling layer for each view can be concatenated to form a single output. The concatenated output can be input into, for example, two fully connected (FC) layers. In one embodiment, the first FC layer can include, for example, 256 units (neurons) and an example ReLu activation function. In one embodiment, the second FC layer can include, for example, two units (neurons) and an example Softmax probability function. The two units can correspond to classification of the input echocardiograms as normal or RHD. The output of the two units can be input to the, for example, Softmax probability function. The output of the Softmax probability function can be a predictive RHD score indicating a probability that the patient has RHD or a binary label of RHD vs. normal.

According to one example, the 3D CNNs can be trained to minimize, for example, a binary cross-entropy loss function using the example Adam optimization algorithm with an example learning rate of 0.0001 over, for example, 350 epochs and an example batch size of 64. The 3D CNNs can be used to analyze ventricular systole frames as volume data (3D data) to assess RHD, wherein the temporal component of the echocardiogram is processed as a dimension of the data. It can be understood that the multi-view 3D CNN described herein is a non-limiting example, and the method of integrating multiple views (such as A4CC and PLAXC views) to detect RHD can be extended to various deep learning architectures. In addition, information from each view can be analyzed separately or together, and integration can occur at different network depths, prior to classification, or during score fusion.

In one embodiment, the RHD ensemble model can be used to process echocardiograms acquired over multiple heart beats from multiple views (e.g., A4CC and/or PLAXC views). The echocardiograms can thus include frames from multiple ventricular systole periods. The frames from each ventricular systole period can be homogenized and processed by the RHD ensemble model to determine a final predictive RHD score.

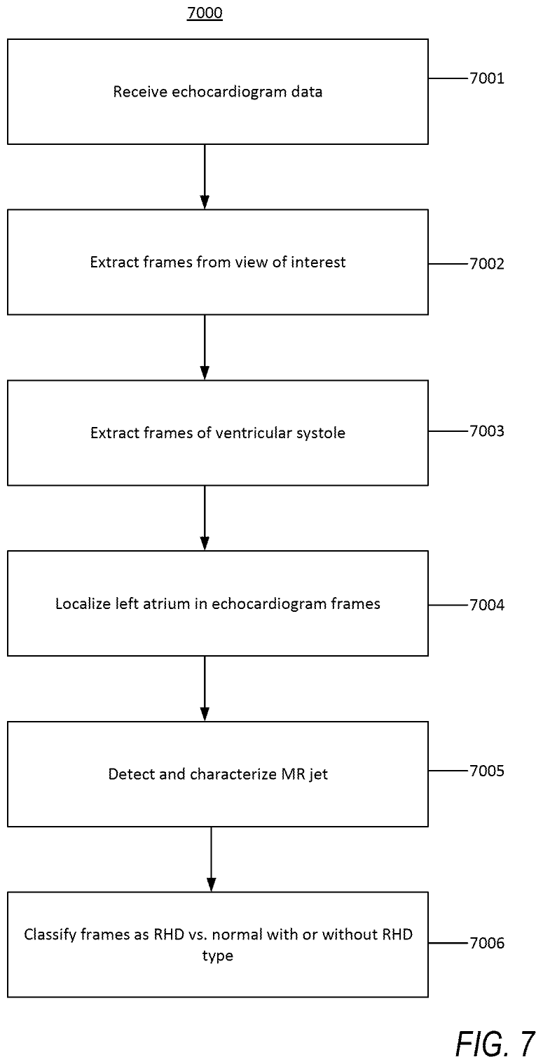

FIG. 7 is a flow chart of a method 7000 for detecting RHD based on MR jet characterization, according to one embodiment of the present disclosure. The method can include receiving echocardiogram data in step 7001. The echocardiogram data can include video data. In one embodiment, the method 7000 can be used to analyze Doppler or color Doppler echocardiograms, which can indicate the flow of blood through the heart. In color Doppler echocardiograms, the direction of blood flow can be represented by different colors. In step 7002, the echocardiograms can be analyzed to extract one or more views of interest. Step 7002 can be executed as is described herein with reference to step 1002 of method 1000. The view of interest can be, for example, the A4CC view and the PLAXC view. In step 7003, the echocardiograms from the one or more views of interest can be analyzed to extract frames corresponding to ventricular systole. Step 7003 can be executed as is described herein with reference to step 1003 of method 1000. In step 7004, the left atrium can be localized in the echocardiogram. In one embodiment, the left atrium can be localized in step 7004 using the localization model described with reference to step 5004 of FIG. 5. In step 7005, the MR jet can be identified and characterized based on the homogenized echocardiogram data. In step 7006, the echocardiograms can be classified based on the MR jet characteristics. In one embodiment, the classification of the echocardiograms in step 7006 can further be based on patient demographic and/or clinical information and/or additional information from valvular heart conditions such as aortic valve regurgitation (AR) and/or image-based information obtained from deep-learning model(s). In one embodiment, the MR jet can be identified and characterized based on an entire frame corresponding to ventricular systole rather than the localized left atrium. A label and/or probability value with or without RHD type can be assigned to the echocardiogram as an output. The label can indicate whether the echocardiogram is indicative of the patient having RHD based on the MR jet. In one embodiment, data that is acquired, generated, or output in any steps of the method 7000 can be output by a device, e.g., via a user interface. For example, the MR jet characteristics that are identified in step 7005 can be output via a user interface for further clinical analysis. In one embodiment, the output of the MR jet characteristics can include a visualization of the MR jet and/or the MR jet characteristics.

The MR jet can be characterized in step 7005 using image analysis of the localized echocardiogram data from the extracted frames by an MR jet analysis model. In one embodiment, the MR jet analysis model can be a machine learning image classification model. In one embodiment, the MR jet analysis model can be used to detect MR jet regions inside the left atrium during ventricular systole in one or more views. In one embodiment, the MR jet analysis model can be used to identify, for example, the largest connected MR jet region inside the left atrium during ventricular systole in one or more views. The one or more views can include, for example, the A4CC view and the PLAXC view. The largest connected MR jet region can be identified, for example, based on the intensity of color of the MR jet in a color Doppler echocardiogram and/or a proximity of the MR jet to the mitral valve, or it can be identified through at least one machine learning classifier. In one embodiment, the MR jet analysis model can be used to identify an echocardiogram frame for each view with, for example, the largest connected MR jet region. In one embodiment, the MR jet analysis model can be used to fit, for example, an oriented bounding box around the MR jet region in the echocardiogram frame. The oriented bounding box can be used to determine, for example, the length, width, area, and perimeter of the MR jet. In one embodiment, the MR jet analysis model can extract MR jet characteristics based on the echocardiogram frames, the MR jet characteristics including, but not limited to, size descriptors (e.g., jet area, length), a ratio between the atrium area and the MR jet size (e.g., length), statistical measures related to the MR jet intensity (e.g., mean, maximum, minimum, median, standard deviation, skewness, kurtosis, entropy), and duration of the MR jet. The duration of the MR jet can be determined, for example, based on a number of extracted frames corresponding to ventricular systole. In general, the MR jet characteristics can be any characteristics associated with the morphology and/or physiology of the MR jet such as pattern, velocity, shape, location, and duration of the MR jet. In one embodiment, the MR jet characteristics can be determined for each echocardiogram view.

In one embodiment, RHD detection and the classification of the echocardiograms in step 7006 can be based on, for example, the MR jet characteristics determined in step 7005, patient demographic and/or clinical information and/or the information from valvular heart conditions such as aortic valve regurgitation (AR) and/or image-based information obtained from deep learning model(s). In one embodiment, the echocardiograms can be classified in step 7006 using at least one RHD classifier. The RHD classifier can be any machine learning classifier, such as a support vector machine (SVM). In one embodiment, for example, Pratt scaling can be applied to an output of the example SVM to calibrate the output probabilities using a logistic regression model. The logistic regression model can be determined based on echocardiogram data used to train the example SVM. The input to the RHD classifier can be one or more characteristics of the MR jet, patient demographic and/or clinical information and/or the information from valvular heart conditions such as AR (aortic valve regurgitation) and/or image-based information obtained from deep learning model(s). The output of the RHD classifier can be a binary label and/or probability value of whether the echocardiogram is normal or indicative of RHD with/without RHD type based on a combination of at least one of MR jet characteristics, patient demographic and/or clinical information, the information from valvular heart conditions such as AR, and/or image-based information obtained from deep learning model(s). In one example, the combination of MR jet characteristics can include, for example, a maximum MR jet length in the PLAXC view, a ratio of the MR jet length to the atrium area in the PLAXC view, an MR jet duration in the A4CC view, a skewness of the MR jet velocity in the PLAXC view, a maximum MR jet length across one or more views, a skewness of the MR jet velocity in the A4CC view, a maximum ratio of the MR jet length to the atrium area across one or more views, an area of the atrium in the PLAXC view, and a standard deviation of the MR jet velocity in the A4CC view. The RHD classifier can be used to evaluate echocardiograms based on at least one of MR jet characteristics, patient demographic and/or clinical information, the information from valvular heart conditions such as AR, and/or image-based information obtained from deep learning model(s).

In one embodiment, the method 7000 can be modified to detect and characterize an AR jet. AR can occur in the left ventricle during diastole. In one embodiment, the method 7000 can be modified so that frames of ventricular diastole are extracted in step 7003. The left ventricle can be localized in step 7004. The frames can then be analyzed to detect and characterize an AR jet. The morphological and/or physiological characteristics of the AR jet can include the types of characteristics of the MR jet described herein. The RHD classifier can classify frames based on morphological and/or physiological characteristics of the MR jet and/or of the AR jet.

In one embodiment, the RHD classifier can be evaluated using k-fold cross-validation. According to one example, the RHD classifier can be evaluated, for example, using 6-fold cross validation with five folds for validation and one fold for testing.

It can be appreciated that the machine learning models described herein are presented as non-limiting examples of the model architectures and types that can be used for the systems and methods of the present disclosure. In one non-limiting example, the view identification model can be residual network ResNet50 CNN, followed by a 7×7 average pooling layer, a fully connected layer of 512 units, and a final output layer. In one example, an image classification CNN described herein can be a residual network ResNet50 CNN, followed by a 7×7 average pooling layer, a fully connected layer of 512 units, and a final output layer. Any of the steps described herein can be performed using one or more machine learning models or can be combined to be performed by one or more machine learning models.

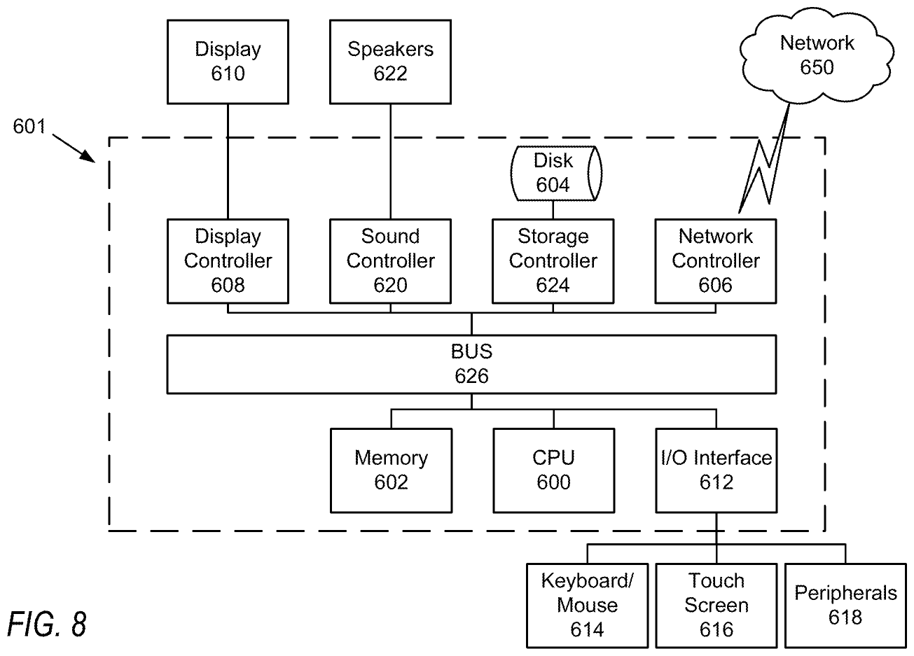

In one embodiment, the echocardiogram homogenization and analysis for RHD prediction, including the use of machine learning models, described herein can be implemented by a computing device or system, such as the device or system as illustrated in FIG. 8. In one embodiment, the computing device can be connected to or in communication with an ultrasound machine. The computing device can receive the echocardiogram data from an ultrasound machine. Next, a hardware description of a device 601 according to exemplary embodiments is described with reference to FIG. 8. In FIG. 8, the device 601 includes processing circuitry, as discussed above. The processing circuitry includes one or more of the elements discussed next with reference to FIG. 8. In FIG. 8, the device 601 includes a CPU 600 which performs the processes described above/below. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 8 can also include a network controller 606, such as an Intel Ethernet PRO network interface card from Intel Corporation of America, for interfacing with network 650, and to communicate with the other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA GeForce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music. In one embodiment, the device 601 can include a data acquisition (DAQ) controller to receive data corresponding to the ultrasound images.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Embodiments of the present disclosure may also be set forth in the following parentheticals.
(1) A method for detecting rheumatic heart disease (RHD) based on at least an echocardiogram, the method comprising: receiving, via processing circuitry, echocardiogram data; extracting, via the processing circuitry, first frames corresponding to at least one echocardiogram view from the echocardiogram data; extracting, via the processing circuitry, second frames corresponding to ventricular systole from the first frames corresponding to the at least one echocardiogram view; and determining, via at least one machine learning model executed by the processing circuitry, an RHD risk score based on the second frames corresponding to ventricular systole.
(2) The method of (1), wherein the echocardiogram data includes Doppler echocardiogram data, color Doppler echocardiogram data, or B-mode ultrasound data.
(3) The method of (1) to (2), wherein the at least one echocardiogram view includes an apical 4-chamber (A4CC) view and/or a parasternal long axis (PLAXC) view.
(4) The method of (1) to (3), wherein determining the RHD risk score via the at least one machine learning model includes assigning greater weight to the second frames corresponding to ventricular systole than to frames corresponding to cardiac phases outside of ventricular systole.
(5) The method of (1) to (4), further comprising identifying and characterizing a mitral valve regurgitation (MR) jet in the second frames corresponding to ventricular systole and/or an aortic valve regurgitation (AR) jet.
(6) The method of (1) to (5), wherein the determining the RHD risk score is based on morphological and/or physiological characteristics of the MR jet and/or morphological and/or physiological characteristics of the AR jet.
(7) The method of claim (1) to (6), wherein the morphological and/or physiological characteristics of the MR jet include at least one of a size descriptor, a shape descriptor, a ratio between an atrium area and an MR jet size, statistical measures related to MR jet intensity or velocity, and duration of the MR jet.
(8) The method of (1) to (7), wherein the determining the RHD risk score is based on patient demographic and/or clinical information, information from other valvular heart conditions, and/or image-based information obtained from a deep learning model.
(9) The method of (1) to (8), further comprising localizing frame data corresponding to at least one atrium region in the second frames corresponding to ventricular systole.
(10) The method of (1) to (9), wherein determining the RHD score via the at least one machine learning model includes assigning greater weight to the frame data corresponding to at least one atrium region in the second frames than to frame data corresponding to regions outside of the at least one atrium region in the second frames.
(11) The method of (1) to (10), wherein the at least one machine learning model is an ensemble model including at least one machine learning classifier, and outputs of the at least one machine learning classifier are fused to determine the RHD risk score.
(12) The method of (1) to (11), further comprising classifying a type of RHD based on the second frames corresponding to ventricular systole.
(13) A non-transitory computer-readable storage medium for storing computer-readable instructions that, when executed by a computer, cause the computer to perform a method for detecting rheumatic heart disease (RHD) based on at least an echocardiogram, the method comprising receiving echocardiogram data; extracting first frames corresponding to at least one echocardiogram view from the echocardiogram data; extracting second frames corresponding to ventricular systole from the first frames corresponding to the at least one echocardiogram view; and determining, via at least one machine learning model, an RHD risk score based on the second frames corresponding to ventricular systole.
(14) The non-transitory computer-readable storage medium of (13), wherein the echocardiogram data includes Doppler data, color Doppler echocardiogram data, or B-mode ultrasound data.
(15) The non-transitory computer-readable storage medium of (13) to (14), wherein the at least one echocardiogram view includes an apical 4-chamber (A4CC) view and/or a parasternal long axis (PLAXC) view.
(16) The non-transitory computer-readable storage medium of (13) to (15), further comprising identifying and characterizing a mitral valve regurgitation (MR) jet in the second frames corresponding to ventricular systole and/or an aortic valve regurgitation (AR) jet.
(17) The non-transitory computer-readable storage medium of (13) to (16), wherein the determining the RHD risk score is based on morphological and/or physiological characteristics of the MR jet and/or morphological and/or physiological characteristics of the AR jet.
(18) The non-transitory computer-readable storage medium of (13) to (17), wherein the morphological and/or physiological characteristics of the MR jet include at least one of a size descriptor, a shape descriptor, a ratio between an atrium area and an MR jet size, statistical measures related to MR jet intensity or velocity, and duration of the MR jet.
(19) The non-transitory computer-readable storage medium of (13) to (18), further comprising localizing frame data corresponding to at least one atrium region in the second frames corresponding to ventricular systole.
(20) An apparatus for detecting rheumatic heart disease (RHD) based on at least an echocardiogram, comprising processing circuitry configured to receive echocardiogram data, extract first frames corresponding to at least one echocardiogram view from the echocardiogram data, extract second frames corresponding to ventricular systole from the first frames corresponding to the at least one echocardiogram view, and determine, via at least one machine learning model, an RHD risk score based on the second frames corresponding to ventricular systole.

Obviously, numerous modifications and variations of the present invention are possible in light of the above teachings. It is therefore to be understood that within the scope of the appended claims, the invention may be practiced otherwise than as specifically described herein.

1. A method for detecting rheumatic heart disease (RHD) based on at least an echocardiogram, the method comprising:
receiving, via processing circuitry, echocardiogram data;
extracting, via the processing circuitry, first frames corresponding to at least one echocardiogram view from the echocardiogram data;
extracting, via the processing circuitry, second frames corresponding to ventricular systole from the first frames corresponding to the at least one echocardiogram view; and
determining, via at least one machine learning model executed by the processing circuitry, an RHD risk score based on the second frames corresponding to ventricular systole.

2. The method of claim 1, wherein the echocardiogram data includes Doppler echocardiogram data, color Doppler echocardiogram data, or B-mode ultrasound data.

3. The method of claim 1, wherein the at least one echocardiogram view includes an apical 4-chamber (A4CC) view and/or a parasternal long axis (PLAXC) view.

4. The method of claim 1, wherein determining the RHD risk score via the at least one machine learning model includes assigning greater weight to the second frames corresponding to ventricular systole than to frames corresponding to cardiac phases outside of ventricular systole.

5. The method of claim 1, further comprising identifying and characterizing a mitral valve regurgitation (MR) jet in the second frames corresponding to ventricular systole and/or an aortic valve regurgitation (AR) jet.

6. The method of claim 5, wherein the determining the RHD risk score is based on morphological and/or physiological characteristics of the MR jet and/or morphological and/or physiological characteristics of the AR jet.

7. The method of claim 6, wherein the morphological and/or physiological characteristics of the MR jet include at least one of a size descriptor, a shape descriptor, a ratio between an atrium area and an MR jet size, statistical measures related to MR jet intensity or velocity, and duration of the MR jet.

8. The method of claim 1, wherein the determining the RHD risk score is based on patient demographic and/or clinical information, information from other valvular heart conditions, and/or image-based information obtained from a deep learning model.

9. The method of claim 1, further comprising localizing frame data corresponding to at least one atrium region in the second frames corresponding to ventricular systole.

10. The method of claim 9, wherein determining the RHD score via the at least one machine learning model includes assigning greater weight to the frame data corresponding to at least one atrium region in the second frames than to frame data corresponding to regions outside of the at least one atrium region in the second frames.

11. The method of claim 1, wherein the at least one machine learning model is an ensemble model including at least one machine learning classifier, and outputs of the at least one machine learning classifier are fused to determine the RHD risk score.

12. The method of claim 1, further comprising classifying a type of RHD based on the second frames corresponding to ventricular systole.

13. A non-transitory computer-readable storage medium for storing computer-readable instructions that, when executed by a computer, cause the computer to perform a method for detecting rheumatic heart disease (RHD) based on at least an echocardiogram, the method comprising:
receiving echocardiogram data;
extracting first frames corresponding to at least one echocardiogram view from the echocardiogram data;
extracting second frames corresponding to ventricular systole from the first frames corresponding to the at least one echocardiogram view; and
determining, via at least one machine learning model, an RHD risk score based on the second frames corresponding to ventricular systole.

14. The non-transitory computer-readable storage medium of claim 13, wherein the echocardiogram data includes Doppler data, color Doppler echocardiogram data, or B-mode ultrasound data.

15. The non-transitory computer-readable storage medium of claim 13, wherein the at least one echocardiogram view includes an apical 4-chamber (A4CC) view and/or a parasternal long axis (PLAXC) view.

16. The non-transitory computer-readable storage medium of claim 13, further comprising identifying and characterizing a mitral valve regurgitation (MR) jet in the second frames corresponding to ventricular systole and/or an aortic valve regurgitation (AR) jet.

17. The non-transitory computer-readable storage medium of claim 16, wherein the determining the RHD risk score is based on morphological and/or physiological characteristics of the MR jet and/or morphological and/or physiological characteristics of the AR jet.

18. The non-transitory computer-readable storage medium of claim 17, wherein the morphological and/or physiological characteristics of the MR jet include at least one of a size descriptor, a shape descriptor, a ratio between an atrium area and an MR jet size, statistical measures related to MR jet intensity or velocity, and duration of the MR jet.

19. The non-transitory computer-readable storage medium of claim 13, further comprising localizing frame data corresponding to at least one atrium region in the second frames corresponding to ventricular systole.

20. An apparatus for detecting rheumatic heart disease (RHD) based on at least an echocardiogram, comprising:
processing circuitry configured to
receive echocardiogram data,
extract first frames corresponding to at least one echocardiogram view from the echocardiogram data,
extract second frames corresponding to ventricular systole from the first frames corresponding to the at least one echocardiogram view, and
determine, via at least one machine learning model, an RHD risk score based on the second frames corresponding to ventricular systole.
